(19) United States
(12) Patent Application Publication
(10) Pub. No.: US 2023/0165900 A1
(43) Pub. Date: Jun. 1, 2023

(54) USE OF HUMAN DECIDUAL MESENCHYMAL STEM CELL CULTURING SUPERNATANTS

(71) Applicant: Biospring Medical Co., Ltd., New Taipei City (TW)

(72) Inventor: Chu Li-Li, New Taipei City (TW)

(73) Assignee: Biospring Medical Co., Ltd, New Taipei City (TW)

(21) Appl. No.: 17/579,595

(22) Filed: Jan. 20, 2022

(30) Foreign Application Priority Data
Nov. 26, 2021 (CN) PCT/CN2021/133606

(51) Int. Cl. A61K 35/28; A61P 9/10; C12N 5/0775

(52) U.S. Cl. CPC A61K 35/28; A61P 9/10; C12N 5/0663

(57) ABSTRACT

Disclosed herein is the cell culturing supernatant from human decidual mesenchymal stem cell culturing that are therapeutic to a subject having an ischemic disease, such as, diabetic foot ulcer. Through the establishment of a mouse hindlimb ischemia model, treatment with cell culturing supernatant from human decidual mesenchymal stem cell culturing can improve the blood supply of the hind limbs. In the streptozotocin induced diabetic model, treatment with cell culturing supernatant from human decidual mesenchymal stem cell culturing can also improve the blood supply of the hind limbs. The invention can be applied to diabetic patients to reduce the incidence of amputation by promoting angiogenesis.

### CROSS-REFERENCES TO RELATED APPLICATIONS

The present application claims priority to the International Patent Application No. PCT/CN2021/133606 filed on 2021 Nov. 26, which is incorporated herein by reference in its entirety.

### FIELD OF THE INVENTION

The present invention discloses the use of cell culturing supernatant of human decidual mesenchymal stem cell, which can be used to prevent or treat ischemic diseases, such as diabetic foot ulcer.

### BACKGROUND OF THE INVENTION

Diabetes Mellitus (DM) has been one of the top ten causes of death in the world. According to the statistics from National Health Promotion Administration of Taiwan, there are approximately 2 million DM patients in Taiwan. In 2016, 9,960 patients died of DM, which has increased by 4.5% over last year.

According to the statistics, 15-25% of diabetic patients suffer from diabetic foot ulcer (DFU) during their lifetime. DFU is considered to be the most serious problem in the pathogenesis of diabetic and the main reason for the hospitalization of DM patients.

So far, due to the dysfunction of angiogenesis caused by diabetes, a large number of DM patients have to amputate their limbs, which cause serious complications.

Mesenchymal stromal cells (MSCs) have been widely studied as a reliable cell source for the treatment on regenerative medicine. MSCs can promote the angiogenesis through paracrine effect. Also, evidence shows that MSCs transplantation can accelerate wound healing, improve clinical symptoms, and avoid amputation.

Under normal circumstances, the inner walls of blood vessels of healthy people are smooth. If they are injured by non-specific damage, such as high blood pressure, cigarette hydrocarbons, cholesterol, high blood sugar, inflammation, trauma and other factors, fatty plaques will accumulate in injuries, causing damage to vascular endothelial cells, allowing lipids to penetrate into the middle layer of vascular endothelium. After the lipids are peroxidized, macrophages would be induced to activate the immune response and trigger a cascade of cytokine secretion, resulting in improper proliferation of smooth muscles in the middle layer of the blood vessel wall and continuous thicken of fatty plaques. In a long-term, ulcers, bleeding or calcification will occur in the middle layer of the blood vessels, and finally form the fibrous plaques, which will make the surface of the blood vessels uneven, and then interact with the platelets to form thrombi, leading to angiemphraxis.

As the result, the below tissues and the blockage site cannot get sufficient nutrients and oxygen, which leads to the ischemia or gangrene. Such pathological changes refer to as peripheral arterial occlusion disease (PAOD).

If diabetic patients have poor blood glucose control for a long time, it will accelerate arteriosclerosis and thicken the basal layer of the blood vessel wall, resulting in poor tissue oxygen permeability, enhanced blood coagulation, and prone to thrombosis, which will further cause luminal stenosis, obstruction, and hind limb ischemia.

### SUMMARY OF THE INVENTION

In view of the above technical circumstances, the present invention provides a human decidual mesenchymal stem cell culturing supernatant, which is prepared from the following steps: (a) providing a serum-free stem cell medium for subculture of stem cells, wherein the serum-free stem cell medium comprises: a serum-free stem cell culture medium; 0.9 to 1.1% insulin-transferrin-selenium; and 9 to 11 ng/ml epidermal growth factor; (b) culturing the serum-free stem cell for 4 to 12 days to obtain the conditioned medium; (c) collecting the cultured serum-free stem cell conditioned medium from step (b) and adding 40 to 80 mg/ml trehalose and 10 to 30 mg/ml dextran to the cultured serum-free stem cell conditioned medium to obtain the antifreeze contained conditioned medium; and (d) filtering the antifreeze contained conditioned medium from step (c) to obtain the human decidual mesenchymal stem cell culturing supernatant.

On the other hand, the present invention provides a method of treating or preventing ischemic disease, comprising administrating an effective amount of a human decidual mesenchymal stem cell culturing supernatant into the subject suffering from ischemic disease, wherein the human decidual mesenchymal stem cell culturing supernatant is defined previously.

In one embodiment of the present invention, the ischemic disease refers to diabetic foot ulcer.

### BRIEF DESCRIPTION OF THE DRAWINGS

FIG. 1 shows the Doppler blood flow image in mouse diabetic hindlimb ischemia model treated by human decidual mesenchymal stem cell culturing supernatant in intramuscular injection and intraperitoneal injection. Picture of the first row is the Doppler blood flow image before the surgery of femoral artery ligation performed. The second row is the image after the surgery of femoral artery ligation finished.

FIG. 2 shows the number of extracellular vesicles in the human decidual mesenchymal stem cell culturing supernatant.

FIG. 3 shows the blood perfusion ratio of ischemic leg treated by human decidual mesenchymal stem cell culturing supernatant in intramuscular injection and intraperitoneal injection.

### DETAILED DESCRIPTION OF THE INVENTION

As used herein, “a,” “an,” “the,” “at least one,” and “one or more” are used interchangeably.

The present invention provides a human decidual mesenchymal stem cell culturing supernatant for preventing or treating ischemic diseases, comprises administrating an effective amount of the human decidual mesenchymal stem cell culturing supernatant; and a pharmaceutically acceptable salt, carrier, diluent or excipient.

A human decidual mesenchymal stem cell culturing supernatant is prepared from the following steps: providing a serum-free stem cell medium for subculturing of stem cells, wherein the serum-free stem cell medium comprises: a serum-free stem cell culture medium; 0.9 to 1.1% insulin-Transferrin-selenium; and 9 to 11 ng/ml epidermal growth factor; culturing the serum-free stem cell for 4 to 12 days to obtain the conditioned medium; collecting the cultured serum-free stem cell conditioned medium and adding 40 to 80 mg/ml trehalose and 10 to 30 mg/ml dextran to the cultured serum-free stem cell conditioned medium to obtain the antifreeze contained conditioned medium; and filtering the antifreeze contained conditioned medium to obtain the human decidual mesenchymal stem cell culturing supernatant.

The effective amount of the human decidual mesenchymal stem cell culturing supernatant can be defined by the weight of the freeze-dried powder form of the supernatant. Preferably, in the embodiment of the present invention, the weight of the supernatant of freeze-dried powder is 0.1879 gram weight, 0.2200 gram weight, and 0.2470 gram weight.

The effective amount of the human decidual mesenchymal stem cell culturing supernatant can be defined by the number of extracellular vesicles. Preferably, in the embodiment of the present invention, the total number of extracellular vesicles in the supernatant cultured by the human decidual mesenchymal stem cells is 1×10⁶ to 1×10¹¹ particles per milliliter.

In another embodiment of the present invention, the total number of extracellular vesicles in the supernatant cultured by the human decidual mesenchymal stem cells is 1×10⁷ to 1×10¹⁰ particles per milliliter.

The present invention provides a method of treating or preventing ischemic disease, comprising administrating an effective amount of a human decidual mesenchymal stem cell culturing supernatant into the subject suffering from ischemic disease, wherein the human decidual mesenchymal stem cell culturing supernatant is defined previously.

In one embodiment of the present invention, the ischemic disease refers to diabetic foot ulcer.

### DESCRIPTION OF EMBODIMENTS

It should be understood that the detailed description of the embodiments is to illustrate the preferred embodiments of the present invention, and is not intended to limit the present invention to certain embodiments. It should be noted that the present invention is intended to cover all alternative embodiments within the same spirit and scope of the present invention. Some non-essential modifications and adjustments made by others based on the concept of the present invention still belong to the protection scope of the present invention

### EXAMPLE 1

### Cell Culture of Human Decidual Mesenchymal Stem Cell

The present invention was performed in the clean room. Aseptic processing was applied for subculturing the human decidual mesenchymal stem cell without any serum ingredient. The conditioned medium was refreshed with fresh medium every three days and cultured for 4 to 12 days. The medium comprises MCDB201 formula, insulin transferrin selenium (ITS) with a concentration ranging from 0.9 to 1.1% and epidermal growth factor with a concentration ranging from 9 to 11 ng/ml (epidermal growth factor, EGF).

The medium and the additives formula of the present invention were all non-animal, serum-free, phenol red-free and chemical defined. Hence, there is no risk of infection or allergic reactions induced by the animals or serum substances to the human body. The supernatant can be directly collected when the stem cell reached the optimal state for subsequent application of the present invention.

The stem cells well sub-cultured at a density of 1×10⁴/cm² in T175 Flask. When the density of stem cell covered above 90%, the conditioned medium with a total protein concentration of 200˜300 μg/ml were ready to be collected and freeze-dried.

### EXAMPLE 2

### Freeze-Drying Preparation of Supernatant Powder of Human Decidual Mesenchymal Stem Cell

In order to maintain the stability of the active components in the conditioned medium, 60 mg/ml trehalose and 20 mg/ml dextran were added and stirred evenly to prepare of the antifreeze contained conditioned medium. Then, the antifreeze contained conditioned medium was filtered by the filter membrane with a pore size of 0.22 μM or less. Fill and filtered solution in a low-temperature resistant container at 2 ml and to finish the preparation of the antifreeze contained conditioned medium.

The freeze dryer had to be pre-cooled to −30° C. first (it regarded for 1 to 2 hours). The samples were placed in the vacuum box and the temperature of the box was cooled to −50° C. within 2 to 3 minutes (rapidly frozen into a solid state). Then, the machine was initiated to pump negative pressure to the box until 200 Torr, the negative pressure was kept for 60 hours to make the ice sublime into water vapor, which removed the moisture of the samples. Then, the temperature of the box was raised to 10° C. and the freeze-dried powder of sample were collected and storage in the can from −20° C. to −80° C.

### EXAMPLE 3

### Number of Extracellular Vesicles in the Human Decidual Mesenchymal Stem Cell Culturing Supernatant

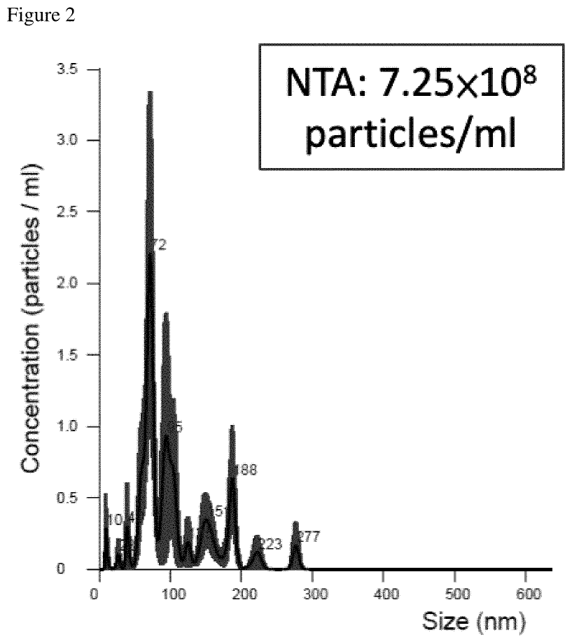

According to the results of the number of extracellular vesicles shown in FIG. 2, the supernatant was analyzed by Nanoparticle Tracking Analysis (NTA). FIG. 2 showed that there were 7.25×10⁸/ml extracellular vesicles. Since the lyophilized powder was dried from 2 ml of the supernatant, there were about 1.45×10⁹ extracellular vesicles of each treatment of supernatant freeze-dried powder.

Other embodiments of the present invention, the total number of extracellular vesicles in the human decidual mesenchymal stem cell culturing supernatant were 9.7×10⁷, 8.6×10⁸, 1.1×10⁹, 1.5×10⁹, 2.3×10⁹, and 1.45×10⁹ respectively.

### EXAMPLE 4

### Streptozotocin Diabetic Mouse Model

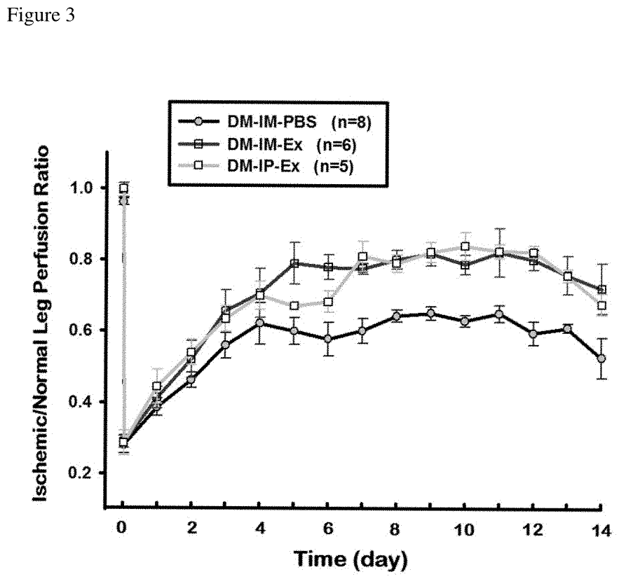

According to the results of supernatant treatment of the hindlimb ischemia model of diabetic mice shown in FIG. 3, streptozotocin was used to induce the symptoms of diabetes, then the hind limb ischemia was induced by the surgery and the treatment were given through intramuscular injection and intraperitoneal injection.

C57BL/6J mice were anesthetized by 2.5% isoflurane gas for carrying out the surgeon of femoral artery ligation. After the surgery, 0.75% bupivacaine (100 μL) was injected intraperitoneally for three days to relieve pain. The Doppler blood flow image were performed to record and analyze the blood flow of the hind limbs of the mice. When the mice have not yet sutured the skin after the femoral artery ligation, the supernatant freeze-dried powder was re-dissolved with 400 μL phosphate-buffered saline (PBS), and the solution were injected intramuscularly or intraperitoneally.

The mouse model of streptozotocin induced diabetes mellitus was to inject 40 mg/kg of streptozotocin intraperitoneally for 5 consecutive days from the first day of the experiment. On the 14th day of the experiment, the mice were fasted and were given Roche (Diastix) test to detect urine glucose level. When the urine glucose level was higher than 3 for two consecutive days, the values of blood glucose were measured after 6 hours of fasting. When the values of blood glucose result were greater than 300 mg/dl, the diabetic induction worked. Then, the surgeon of femoral artery ligation was carried out and defined as day 0 of the animal experiment for giving the treatment of freeze-fried powder of cell culturing supernatant of human decidual mesenchymal stem cell.

### EXAMPLE 5

### Doppler Blood Flow Image Analysis

C57BL/6J mice were anesthetized by 2.5% isoflurane gas for carrying out the surgery of femoral artery ligation. After the surgery, 0.75% bupivacaine (100 μL) were injected intraperitoneally for three days to relieve pain. The Doppler blood flow image were performed to record and analyze the blood flow of the hind limbs of the mice.

When the mice have not yet sutured the skin after the femoral artery ligation, the supernatant freeze-dried powder was re-dissolved with 400 μL PBS, and the PBS with the re-dissolved supernatant freeze-dried powder was injected intramuscularly or intraperitoneally. Intramuscular injection was performed on the calf and the inner and outer muscle of the thigh with 60 μL respectively, a total of 180 μL supernatant was injected. Likewise, 180 μL of supernatant was injected intraperitoneally into the abdominal cavity of the mice once after the surgery.

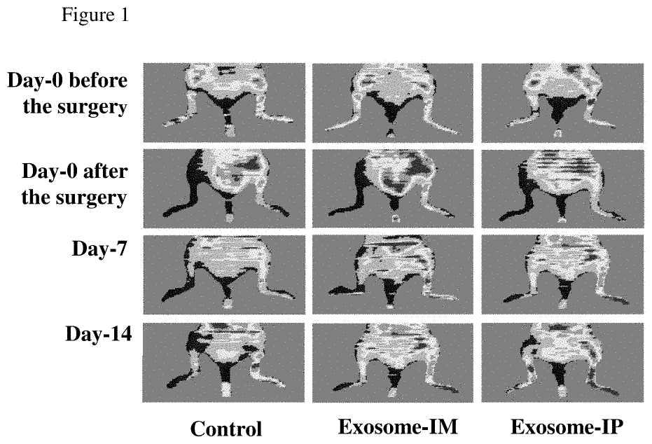

According to the image as shown in FIG. 1, picture of the first row is the Doppler blood flow image before the surgery of femoral artery ligation performed. The second row is the image after the surgery of femoral artery ligation finished. The image with deep color stands for less blood flow and vice versa. The blood flow of the right foot of the mouse was reduced after the surgery, but the blood flow of the right foot of the treated group was higher than that of the control group on the 7th and 14th day.

The Doppler blood flow image data was monitored for 14 consecutive days after the surgery and were quantified as shown in FIG. 3. The results showed that relatively higher blood flow in both the intramuscular injection and intraperitoneal injection treated groups were observed compared to the control group.

Overall, diabetic mice have poorer blood flow recovery ability than healthy mice. In this state, administration of human decidual mesenchymal stem cell supernatant can effectively improve blood flow. The data were presented as mean±standard deviation. DM: diabetic mellitus mice; IM: intramuscular injection; IP: intraperitoneal injection; EX: human decidual mesenchymal stem cell culturing supernatant.

While the invention has been described and exemplified in sufficient details for those skilled in this art to make and use it, various alternatives, modifications, and improvements should be apparent without departing from the spirit and scope of this invention.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A human decidual mesenchymal stem cell culturing supernatant, which is prepared from the following steps:
(a) providing a serum-free stem cell medium for subculture of stem cells,
wherein the serum-free stem cell medium comprises:
a serum-free stem cell culture medium;
0.9 to 1.1% insulin-transferrin-selenium; and
9 to 11 ng/ml epidermal growth factor;
(b) culturing the serum-free stem cell for 4 to 12 days to obtain the conditioned medium;
(c) collecting the cultured serum-free stem cell conditioned medium from step (b) and adding 40 to 80 mg/ml trehalose and 10 to 30 mg/ml dextran to the cultured serum-free stem cell conditioned medium to obtain the antifreeze contained conditioned medium; and
(d) filtering the antifreeze contained conditioned medium from step (c) to obtain the human decidual mesenchymal stem cell culturing supernatant.

2. The human decidual mesenchymal stem cell culturing supernatant of claim 1, wherein the number of extracellular vesicles in the human decidual mesenchymal stem cell culturing supernatant is 1×10⁶ to 1×10¹¹ particles per milliliter.

3. The human decidual mesenchymal stem cell culturing supernatant of claim 2, wherein the number of extracellular vesicles in the human decidual mesenchymal stem cell culturing supernatant is 1×10⁷ to 1×10¹⁰ particles per milliliter.

4. The human decidual mesenchymal stem cell culturing supernatant of claim 1, wherein the serum-free stem cell culture medium is MCDB201 culture medium.

5. The human decidual mesenchymal stem cell culturing supernatant of claim 1, wherein the human decidual mesenchymal stem cell culturing supernatant is stored through freeze drying.

6. The method of treating or preventing ischemic disease, comprising administrating an effective amount of a human decidual mesenchymal stem cell culturing supernatant into the subject suffering from ischemic disease, wherein the human decidual mesenchymal stem cell culturing supernatant is as claimed in claim 1 or a pharmaceutically acceptable salt, carrier, diluent or excipient.

7. The method of claim 6, wherein the ischemic disease is diabetic foot ulcer.

8. The method of claim 7, wherein the administration is provided through intramuscular injection or intraperitoneal injection.
